SEQUENCE LISTING (4 sequences)

SEQ ID NO: 1 - RNA, 60 nt, Homo sapiens
gaucacgggccuuguacacugucccauaggcaccgacaccaauuucagccacuggcucau

SEQ ID NO: 2 - DNA, 60 bp, Homo sapiens
ctctacactaaggggtatgttccctcttgtccctttccctacctttatatttggggtcct

SEQ ID NO: 3 - RNA, 60 nt, Homo sapiens
ucacugagcacuaguccagccaaggggucaguggaggggcccccagaggccccagccccc

SEQ ID NO: 4 - RNA, 60 nt, Homo sapiens
ggcucggcuuggauuauucugcagguucaucucagacccaccuuucagccuuaagcagcc

(19) United States
(12) Patent Application Publication
(10) Pub. No.: US 2008/0300143 A1
(43) Pub. Date: Dec. 4, 2008

(54) ENDOGENOUS ANTISENSE RNA EXPRESSION ANALYSIS SYSTEM

(75) Inventor: Hidenori KIYOSAWA, Tsukuba-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi (JP)

(21) Appl. No.: 12/129,905

(22) Filed: May 30, 2008

(30) Foreign Application Priority Data
May 31, 2007 (JP) 146341/2007

(51) Int. Cl. C40B 30/04; C07H 21/04; C40B 40/08

(52) U.S. Cl. 506/9; 536/24.3; 536/24.31; 506/17

(57) ABSTRACT

Provided is a novel endogenous antisense RNA expression analysis system capable of comprehensively and highly-precisely detecting endogenous antisense RNA including noncoding antisense RNA. A probe set containing one or more probes designed for an antisense strand sequence (Artificial Antisense Sequence: AFAS) under the conditions optimal for hybridization, by artificially defining an antisense strand of known cDNA; a microarray containing the AFAS probe set; detection method of endogenous antisense RNA wherein the microarray and RNA labeling by random priming are combined, and the like.

### TECHNICAL FIELD OF THE INVENTION

The present invention relates to a novel probe capable of detecting an unknown endogenous antisense RNA, particularly, a probe set capable of comprehensively and highly-precisely detecting an endogenous antisense RNA including a noncoding antisense RNA, a microarray immobilizing same, use thereof, and the like.

### BACKGROUND OF THE INVENTION

Antisense RNA is an RNA having a nucleotide sequence complementary to mRNA. Specifically, it is an RNA read from an opposite strand of a DNA strand encoding a sense gene. Sense-antisense RNAs are capable of forming a double-strand and, for example, it is known that a double-stranded RNA is necessary for RNA interference, a double-stranded RNA is involved in the regulation of translation of a protein by a small RNA called microRNA and the like.

About 2,500 pairs of sense-antisense genes have been identified in mouse, which include many noncoding genes that do not encode proteins (non-patent reference 1). About half of the sense-antisense gene pairs is considered to be coding-noncoding gene pairs. Also in human, the presence of about 2,600 pairs of sense-antisense RNA pairs is suggested (non-patent reference 2). However, experimental verification relating to the structures and expressions thereof is limited.

The present inventors and their coworkers have so far developed an oligo DNA chip that distinguishes and analyzes 1947 pairs of sense gene and antisense gene identified in mouse, and comprehensively analyzed the expression of sense-antisense genes (non-patent reference 3). As a result, they have found that more than 90% of the sense-antisense genes are expressed in actual tissues, and that the expression thereof shows tissue specificity. It has also been found that various sizes of RNAs are transcribed from the sense-antisense gene locus, most of the RNAs lack the poly(A) chain and they tend to be accumulated in the nucleus. Furthermore, RNA without the poly(A) chain was also found by analysis of Arabidopsis, and such properties were found to be common to animals and plants.

Nevertheless, the physiological role of such noncoding antisense RNAs has not been elucidated at all.

The exploration of physiological function of noncoding antisense RNA requires comprehensive analysis of the expression. However, a microarray having probes produced by utilizing conventional cDNA sequences can only have probes designed for genes having a cDNA sequence on an antisense strand (i.e., coding RNA), and therefore, fails to detect novel antisense RNAs (particularly noncoding antisense RNAs).

On the other hand, the genome tiling array developed by Affymetrix has evenly tiled probes across a genome sequence, irrespective of the presence of gene information from the cDNA sequence and, in principle, may be able to detect a novel antisense RNA even in the absence of cDNA sequence information. However, the genome tiling array has problems in the property of the probes, since probes are designed evenly and forcedly relative to the genomic DNA sequence, and the background is very high and the noise is large, thus making it difficult to distinguish the signals. Due to its specification, it has many other problems such as inability to distinctly analyze the sense strand and antisense strand and the like.

For an efficient detection of a novel endogenous antisense RNA (particularly noncoding antisense RNA) to be realized, therefore, it is necessary to afford a novel antisense RNA detection method, which is different from conventional cDNA array and genome tiling array designed based on a cDNA sequence.

non-patent reference 1: Kiyosawa et al, Genome Res. 13: 1324-1334 (2003)
non-patent reference 2: Yelin et al, Nat. Biotechnol. 21: 379-386 (2003)
non-patent reference 3: Kiyosawa et al, Genome Research, 15: 463-474 (2005)

### DISCLOSURE OF THE INVENTION

### Problems to be Solved by the Invention

It is therefore an object of the present invention to provide a novel endogenous antisense RNA expression analysis system capable of comprehensive and highly-precise detection of an endogenous antisense RNA including a noncoding antisense RNA.

### Means of Solving the Problems

The present inventors artificially postulated, as a putative antisense RNA, an antisense strand sequence deduced from a known cDNA sequence, designed a probe for the antisense strand sequence (Artificial Antisense Sequence: AFAS) under the same conditions as for designing a probe for general cDNA sequences, i.e., conditions optimal for hybridization, and constructed a microarray containing the probe (AFAS probe) along with a probe for cDNA sequence (sense strand sequence). Furthermore, the present inventors labeled samples from cancer patients by random priming and, using the microarray, comprehensively analyzed the expression of sense-antisense gene pairs in each sample. As a result, they have found that the expression of sense strands of cyclin-dependent kinase 4 (CDK4) and mitogen activation protein kinase 7 (MAPK7) genes known as cancer genes increased in cancer tissues, whereas expression of the corresponding antisense RNAs remarkably decreased in the cancer tissues. These results first demonstrated clearly that the gene expression is regulated by antisense RNA and abnormality in the regulatory mechanism is closely related to diseases, and that endogenous antisense RNA has a physiological function to regulate the expression of a gene encoded by the corresponding sense strand.

The present inventors made further investigation based on these findings, and demonstrated that a means based on a combination of a novel microarray carrying an AFAS probe set and labeling of total RNA including noncoding (non-poly(A)) RNA by random priming is highly useful for the regulation of gene expression by endogenous antisense RNA and elucidation of its biological significance, which resulted in the completion of the present invention.

Accordingly, the present invention relates to the following.

[1] A probe set comprising at least one kind of probe consisting of a nucleic acid comprising an artificial nucleotide sequence capable of hybridizing to an antisense strand sequence deduced from a known cDNA sequence.
[2] The probe set of the above-mentioned [1], wherein at least one kind of the cDNA is free of addition of a poly(A) chain when its antisense strand is transcribed.
[3] The probe set of the above-mentioned [1] or [2], wherein the cDNA is derived from a mammal.
[4] The probe set of the above-mentioned [3], wherein the mammal is a human or a mouse.
[5] The probe set of any of the above-mentioned [1]-[4], further comprising a probe consisting of a nucleic acid comprising a nucleotide sequence capable of hybridizing to a sense strand of the cDNA.
[6] The probe set of any of the above-mentioned [1]-[5], wherein the number of the cDNA is 100 or more.
[7] A microarray comprising a substrate and the probe set of any of the above-mentioned [1]-[6] immobilized thereon.
[8] A method of detecting an endogenous antisense RNA in an RNA-containing sample using the microarray of the above-mentioned [7], which comprises the steps of
(a) labeling RNA in the sample by random priming,
(b) contacting each probe on the microarray with said labeled RNA,
(c) washing away the RNA not bound to the probe, and
(d) detecting the label of the RNA bound to the probe.
[9] The method of the above-mentioned [8], comprising detecting the expression of mRNA transcribed from a sense strand corresponding to the antisense RNA.
[10] The method of the above-mentioned [8] or [9], wherein the sample is derived from a mammal.
[11] The method of the above-mentioned [10], wherein the mammal is a human or a mouse.
[12] The method of the above-mentioned [9] or [10] used for comparison of expression patterns of endogenous antisense RNAs of mammals, comprising subjecting a target animal and a control animal to said steps (a)-(d), and further identifying endogenous antisense RNA showing different expression patterns between the target animal and the control animal, by comparing the obtained expression patterns of endogenous antisense RNAs of the target animal and that obtained of the control animal.
[13]. The method of the above-mentioned [12], wherein the target animal is affected with a given disease, or an animal model of a given disease.

### EFFECT OF THE INVENTION

The AFAS probe set and the microarray carrying same of the present invention can detect an antisense RNA even for a gene without a known cDNA sequence on the antisense strand. Since a probe for AFAS is designed under similar conditions to those generally employed for cDNA sequence of cDNA array, the microarray is superior in the detection sensitivity and precision as compared to genome tiling array. Moreover, by combining the microarray with RNA labeling by random priming, all noncoding antisense RNAs can be detected without fail. As a result, regulation of gene expression by endogenous antisense RNA can be comprehensively analyzed, and the involvement of endogenous antisense RNA in various biological phenomena, such as relationship between abnormalities in the regulatory mechanism and disease and the like, can be elucidated.

### BRIEF DESCRIPTION OF THE DRAWINGS

FIG. 1 shows expression of CDK4 in normal tissue sample by oligo(dT) priming method, wherein a white bar shows expression of sense strand and a black bar shows expression of antisense strand.

FIG. 2 shows expression of CDK4 in colorectal cancer tissue sample by oligo(dT) priming method, wherein a white bar shows expression of sense strand and a black bar shows expression of antisense strand.

FIG. 3 shows expression of CDK4 in normal tissue sample by random priming method, wherein a white bar shows expression of sense strand and a black bar shows expression of antisense strand.

FIG. 4 shows expression of CDK4 in colorectal cancer tissue sample by random priming method, wherein a white bar shows expression of sense strand and a black bar shows expression of antisense strand.

FIG. 5 shows expression of MAPK7 in normal tissue sample by oligo(dT) priming method, wherein a white bar shows expression of sense strand and a black bar shows expression of antisense strand.

FIG. 6 shows expression of MAPK7 in liver cancer tissue sample by oligo(dT) priming method, wherein a white bar shows expression of sense strand and a black bar shows expression of antisense strand.

FIG. 7 shows expression of MAPK7 in normal tissue sample by random priming method, wherein a white bar shows expression of sense strand and a black bar shows expression of antisense strand.

FIG. 8 shows expression of MAPK7 in liver cancer tissue sample by random priming method, wherein a white bar shows expression of sense strand and a black bar shows expression of antisense strand.

### BEST MODE FOR CARRYING OUT THE INVENTION

The probe set of the present invention contains at least one kind of probe consisting of a nucleic acid comprising an artificial nucleotide sequence capable of hybridizing to an antisense strand sequence deduced from a known cDNA sequence. Since the probe set may consist of only one kind of the above-mentioned probe, it is indicated as “the probe (set) of the present invention” in the following, unless limited to a probe set containing two or more kinds of the above-mentioned probe.

The known cDNA is not particularly limited as long as it is a complementary DNA corresponding to RNA, for which the presence of mRNA (including EST) is known, and may be various cDNAs, any cDNA in the EST database (e.g., GenBank, EMBL, DDBJ etc.), or a newly-cloned cDNA.

The number of the target cDNAs is not particularly limited as long as it is 1 or more, and can be appropriately selected depending on the object thereof. For a comprehensive analysis of antisense RNAs, it is preferably 100 or more, more preferably 1000 or more, and particularly preferably 10000 or more. In addition, cDNA can be randomly selected irrespective of the kind of gene. Alternatively, a cDNA corresponding to a gene cluster known to show expression patterns that vary in a disease specific manner (e.g., cancer gene and the like), a cDNA corresponding to a gene cluster known to show expression patterns that vary in correlation with drug toxicity (e.g., phospholipidosis marker gene and the like), or a cDNA corresponding to genes having particular common properties, such as a gene cluster that is expressed in a stem cell- or differentiated cell-specific manner (undifferentiated marker, differentiation marker gene), may be selected.

The origin of the subject cDNA is not particularly limited as long as it is of the same species, and the cDNA may be derived from any living organism. Preferred is a mammal (e.g., human, mouse, rat, monkey, dog, cow, horse, pig, sheep, goat, rabbit, hamster etc.), more preferred is human, mouse, rat, monkey, dog etc., and particularly preferred is human or mouse.

The “sequence” of a known cDNA means a sequence of a sense strand (strand containing the information to be translated into protein), namely, a nucleotide sequence corresponding to mRNA, and the “antisense strand” means a strand complementary to a sense strand.

The “antisense strand sequence deduced” from a known cDNA sequence is a sequence completely complementary to the sequence determined based on the sense strand sequence, and is a sequence postulated artificially whether actual transcription of the antisense strand sequence from a genomic DNA is known.

An RNA detection probe comprising the above-mentioned antisense strand sequence is not particularly limited as long as it is a nucleic acid comprising a nucleotide sequence hybridizable to a target antisense strand sequence under the hybridization conditions usable for general gene expression analyses. Preferably, the probe is a nucleic acid comprising a nucleotide sequence hybridizable to a target antisense strand sequence under stringent conditions. The “stringent conditions” mean the conditions under which only a nucleotide sequence having 95% or more, preferably 96% or more, more preferably 97% or more, particularly preferably 98% or more, most preferably 99% or more homology to a nucleotide sequence completely complementary to the target nucleotide sequence is hybridizable. Those of ordinary skill in the art can easily control the conditions to have desired stringency by appropriately changing the salt concentration of hybridization solution, temperature of hybridization reaction, probe concentration, probe length, number of mismatches, hybridization reaction time, salt concentration of washing solution, temperature of washing step and the like.

The nucleotide length of the nucleic acid probe constituting the probe (set) of the present invention is not particularly limited as long as it can specifically hybridize to an antisense strand sequence deduced from a target cDNA and is, for example, 15 or more, preferably 20 or more, more preferably 25 or more, bases. In consideration of the easiness of synthesis and the like, the probe length is preferably 200 bases or less, more preferably 100 bases or less. While the nucleotide length of each probe may be different, it is more preferably the same.

When the nucleotide length of the probe is shorter than the full-length of the antisense strand sequence, the position of the target antisense strand sequence is not particularly limited. In general, when an about 25-60 mer probe is to be produced for the detection or analysis of mRNA, since RNA is often labeled by oligo(dT) priming, the nucleotide sequence at the 3′ end side of mRNA is often selected as a target. However, the probe (set) of the present invention efficiently detects a noncoding antisense RNA, i.e., antisense RNA free of addition of poly(A). Therefore, random priming is selected for labeling RNA samples, and therefore, a sequence preferable for hybridization can also be appropriately selected from any position on the antisense strand and used as a target sequence of the probe. Two or more probes targeting different positions can also be designed for one antisense strand.

The probe (set) of the present invention is characterized in that at least one kind of the target cDNA is a cDNA free of addition of poly(A) chain to RNA when its antisense strand is transcribed. In conventional microarray for sense-antisense gene expression analysis produced based on a cDNA sequence, a probe for antisense strand is designed and located only on an antisense strand known to have a cDNA sequence, i.e., a sequence known to have a poly(A) chain on the mRNA. The probe (set) of the present invention automatically postulates an antisense sequence (AFAS) irrespective of whether a cDNA sequence is present on an antisense strand and designs a probe for the AFAS. Therefore, it enables detection of an antisense RNA, particularly noncoding antisense RNA, of even a gene not known to have a cDNA, i.e., coding RNA.

While the number of the target cDNAs, in which poly(A) chain is not added to RNA when antisense strand is transcribed, is not particularly limited, it is preferably 2 or more, more preferably 50 or more, particularly preferably 100 or more, for comprehensive analysis of noncoding antisense RNA.

The probe set of the present invention preferably further contains a nucleic acid comprising a nucleotide sequence capable of hybridizing to a sense strand of cDNA.

The “sense strand” means a strand comprising a nucleotide sequence corresponding to mRNA of cDNA as mentioned above, i.e., a strand containing the information to be translated into a protein. The probe for detection of sense RNA (mRNA) is not particularly limited as long as it is a nucleic acid comprising a nucleotide sequence hybridizable to a target sense strand sequence under hybridization conditions usable for general gene expression analyses. Preferably, the probe is a nucleic acid comprising a nucleotide sequence hybridizable to a target sense strand sequence under stringent conditions. Here, the “stringent conditions” are as defined above. Preferable nucleotide length of a probe targeting a sense strand is, for example, the range of a probe targeting the above-mentioned antisense strand. When the nucleotide length of the probe is shorter than the full-length of cDNA, the position of the sense strand sequence to be targeted is not particularly limited, and a sequence preferable for hybridization is appropriately selected and used as mentioned above.

Such nucleic acid probe can also be obtained by chemically synthesizing the complementary strand sequence thereof based on the information of the nucleotide sequence of the antisense strand or sense strand to be targeted, by using a commercially available DNA/RNA automatic synthesizer and the like. In addition, a chip (array) with a solid phased nucleic acid can also be produced by directly synthesizing in situ (on chip) the nucleic acid on a solid phase such as silicone, glass and the like.

Such nucleic acid probe can be provided in a dry state or as a solid in the state of an alcohol precipitate, or can be provided after dissolution in water or a suitable buffer (e.g., TE buffer etc.). When used as a labeling probe, the nucleic acid can be provided after being labeled in advance with any of the above-mentioned labeling substances, or may be provided separately from a labeling substance and labeled when in use. As the labeling substance, for example, radioisotope, enzyme, fluorescent substance, luminescent substance and the like are used. As the radioisotope, for example, [³²P], [³H], [¹⁴C] and the like are used. As the enzyme, a stable enzyme showing high specific activity is preferable and, for example, β-galactosidase, β-glucosidase, alkaline phosphatase, peroxidase, malate dehydrogenase and the like are used. As the fluorescent substance, for example, fluorescamine, fluorescein isothiocyanate and the like are used. As the luminescent substance, for example, luminol, luminol derivative, luciferin, lucigenin and the like are used. Moreover, biotin-(strept)avidin can be used for binding a probe and a labeling agent. In addition, when a nucleic acid to be the probe is to be immobilized on a solid phase, a nucleic acid in a sample can be labeled using a labeling agent similar to those mentioned above.

In a preferable embodiment of the present invention, the probe (set) of the present invention is provided in the form of a microarray wherein the probe is immobilized on a substrate.

Examples of the materials of the substrate include semiconductors such as silicone and the like, inorganic materials such as glass, diamond and the like, films containing a polymer such as polyethylene terephthalate and polypropylene as a main component, and the like. Examples of the form of the substrate include, but are not limited to, slide glass, micro-well plate, micro-beads, fiber and the like.

Examples of the method for immobilizing a probe on a substrate include, but are not limited to, a method comprising introducing a functional group such as amino group, aldehyde group, SH group, biotin and the like into a nucleic acid in advance, introducing a functional group (e.g., aldehyde group, amino group, SH group, streptavidin and the like) that can react with the nucleic acid, on a solid phase, and crosslinking the solid phase and the nucleic acid by a covalent bond between both functional groups, or coating a solid phase with a polycation and immobilizing a polyanionic nucleic acid by utilizing an electrostatic bond, and the like. Examples of the preparation method of microarray include the Affymetrix type wherein a nucleic acid probe is synthesized by a photolithography method synthesizing nucleotide one by one on a substrate (glass, silicone and the like), and the Stanford type wherein a nucleic acid probe prepared in advance is spotted onto a substrate by a microspotting method, an inkjet method, a bubble jet (registered trademark) method and the like. When a probe of 30 mer or more is used, the Stanford type or a combination of the two types is preferable.

The present invention also provides a method of detecting an endogenous antisense RNA in an RNA-containing sample using the microarray of the present invention. The method comprises the steps (a)-(d) below:

(a) labeling RNA in the sample by random priming,
(b) contacting each probe on the microarray with said labeled RNA,
(c) washing away the RNA not bound to the probe, and
(d) detecting the label of the RNA bound to the probe.

The sample containing RNA is prepared from any living organism by a known method. The living organism to be the origin of the sample containing RNA is not particularly limited as long as it is an organism species from which the target sequence of the probe on the microarray of the present invention is derived, such as animal, plant, bacterium, cell and the like. Preferably, the sample is derived from a mammal, more preferably human or mouse. The sample may be derived from a whole living organism or a part thereof. Examples thereof include cells [e.g., hepatocytes, splenocytes, nerve cells, glial cells, pancreatic β cells, bone marrow cells, mesangial cells, Langerhans cells, epidermal cells, epithelial cells, goblet cells, endothelial cells, smooth muscle cells, fibroblast, fiber cells, muscle cells, adipocytes, immune cells (e.g., macrophage, T cell, B cell, natural killer cell, mast cell, neutrophil, basophil, eosinophil, monocyte), megakaryocytes, synovial cells, chondrocytes, osteocytes, osteoblasts, osteoclasts, mammary cells, interstitial cells, and precursor cells, stem cells, cancer cells and the like of these cells] or any tissues where those cells are present [e.g., brain, each moiety of the brain (e.g., olfactory bulb, amygdaloid nucleus, basal ganglia, hippocampus, thalamus, hypothalamus, cerebral cortex, medulla oblongata, cerebellum), spinal cord, eyeball, pituitary gland, stomach, pancreas, kidney, liver, gonad, thyroid gland, gall bladder, bone marrow, adrenal gland, skin, lung, gastrointestinal tracts (e.g., large intestine, small intestine), blood vessel, heart, thymus, spleen, submandibular gland, peripheral blood, prostate, orchis, ovary, placenta, uterus, bone, joint, adipose tissue, skeletal muscle and the like], and the like. As long as RNA to be detected (e.g., RNA that can be a biomarker such as disease marker, toxicity marker, differentiation marker and the like) can be expressed, blood (e.g., peripheral blood), lymphocytes and the like are preferable since they can be recovered quickly and conveniently, and are less-invasive to animals.

Total RNA fraction is prepared from the above-mentioned cell-containing sample by a method known per se, such as guanidine-CsCl ultracentrifugation method, AGPC method and the like. Using a commercially available RNA extraction kit (e.g., RNeasy Mini Kit; manufactured by QIAGEN etc.), a highly pure total RNA can be prepared rapidly and conveniently from a trace amount of a sample.

The obtained total RNA fraction is labeled using a random primer. As the label, radioisotope, fluorescent substance and the like can be used. As the radioactive element, [³²P], [³H], [¹⁴C] and the like can be used, and as the fluorescent substance, Cy3 (registered trade mark), Cy5 (registered trade mark) and the like can be used. Also, biotin-(strept)avidin may be used nonlimitatively for binding RNA and a labeling agent. For example, labeling can be performed as shown below, though nonlimitatively. First, a cDNA harboring a suitable promoter such as T7 promoter and the like is synthesized from a total RNA fraction by reverse transcription reaction using a random primer, and a cRNA is synthesized using an RNA polymerase (labeled cRNA is obtained here by using, as a substrate, mononucleotide labeled with the above-mentioned labeling substance). The labeled cRNA is contacted with the above-mentioned solid-phased probe to perform a hybridization reaction, and the label bound with each probe on the solid phase is quantified to determine the level of expression of each gene. The hybridization reaction can be performed under the above-mentioned “stringent conditions”.

The label can be detected by a method known per se according to the label used.

The present method may include detection of expression of mRNA transcribed from a sense strand corresponding to an antisense RNA.

The method of the present invention further comprises applying the aforementioned steps (a)-(d) to each of a target animal and a control animal, and comparing the obtained expression pattern of an endogenous antisense RNA of the target animal with that of the control animal, based on which an endogenous antisense RNA varying in expression patterns between the target animal and the control animal is identified. As used herein, the target animal may be, but is not limited to, a mammal affected with a given disease, an animal model of a given disease, a given physically or chemically-stressed mammal or the like.

For example, when the target animal is a mammal affected with a given disease, the expression pattern of an endogenous antisense RNA obtained from the animal is compared with that of a sample containing RNA derived from a control animal not affected with the disease, and an endogenous antisense RNA that shows an expression pattern specific to the disease is identified. The given disease is not particularly limited and includes, for example, various diseases such as cancers such as colorectal cancer, liver cancer and the like, mental diseases, immune diseases, inflammatory diseases, metabolic abnormality diseases, neurodegenerative diseases, cardiovascular disorders, cerebrovascular disorders, blood diseases, infections, digestive system diseases, respiratory diseases, urologic diseases and the like, drug toxicity including abnormal lipid metabolism such as phospholipidosis and the like, and the like. As the control animal, moreover, an animal of the same species as the diseased animal and unaffected with the disease (preferably a normal animal) can be used. In the case of human, a tissue sample derived from a healthy subject is particularly difficult to obtain. Thus, a lesion tissue may be used as a disease sample, and a surrounding normal tissue may be used as a control tissue.

A sample containing RNA derived from a diseased animal and a sample containing RNA derived from a target animal may be separately hybridized to the microarray of the present invention, or simultaneously hybridized competitively. In the latter case, by labeling each sample with, for example, a fluorescent dye with a different color (e.g., Cy3, Cy5 etc.), the difference in the expression of each sample can be quantitatively analyzed by scanning analysis of fluorescent colors.

Consequently, antisense RNA that showed significantly varying expression patterns in the diseased animal as compared to the control animal can be selected as an endogenous antisense RNA showing varying expression patterns in a disease-specific manner, i.e., as a marker antisense RNA of the disease. Whether or not the antisense RNA is noncoding can be verified by comparing the expression of the antisense RNA with that obtained when the RNA of the sample is labeled by oligo(dT) priming and treated in the same manner.

Alternatively, when RNAs are collected from differentiated cell and undifferentiated cell (stem cell and the like) and an antisense RNA showing significantly different levels of expression between them can be obtained as a result of the expression analysis in the same manner as above, the RNA can be selected as a differentiation marker (or undifferentiation marker).

Using, as an index, the expression of a disease marker or differentiation (undifferentiation) marker antisense RNA obtained as mentioned above, diseases and differentiation (undifferentiated) state can be examined. That is, a sample containing RNA derived from a test animal (test cell) and a sample containing RNA derived from a control animal (control cell) are each analyzed for the expression of antisense RNA in the same manner as above and using the microarray of the present invention, and the expression of the marker antisense RNA in the test animal (test cell) is compared with that of the control, based on which whether the test animal is affected (or may be affected) with the disease and whether the test cell is differentiated or undifferentiated can be determined.

Alternatively, it is possible to design and prepare a particular probe and a primer capable of amplifying the sequence based on the nucleotide sequence of a marker antisense RNA, and the expression of the antisense RNA can be quantitatively analyzed by Dot blot, Northern blot hybridization or RT-PCR using them.

The present invention is explained in more detail in the following by referring to Examples, which are not to be construed as limitative.

### EXAMPLE 1

### Production of Microarray

Using a gene-specific 60 mer oligo DNA probe and a system of Agilent Technologies Inc., a custom-made microarray was prepared. As the probe, a probe of a gene sequence (sense strand) used for cancer research and its antisense strand probe were placed on the microarray. As for the antisense strand probe, a complementary strand sequence of the cDNA sequence of the gene was prepared, and specific 60 mer was selected and placed on the array. As a result of cDNA sequence analysis, the antisense strand probes also contained antisense strand probes of the gene for which the antisense strand corresponding to the sense strand was not found.

### EXAMPLE 2

### Expression Analysis

Using the microarray chip of Example 1, the expression in a cancer sample was analyzed. As an oligo(dT) label for the sample, a labeling kit and a fluorescence dye (Cy3) for a single-dye method as specified by Agilent Technologies Inc. were used, and a target cRNA was produced and hybridized. For a random priming method, a target cDNA was prepared using a CyScribe First-Strand cDNA Labeling Kit (Amersham) and hybridized. The hybridization and washing were performed according to the protocol recommended by Agilent Technologies Inc.

After the hybridization, the fluorescence intensity on the slide glass was measured by a scanner of Agilent Technologies Inc., and the data was corrected using a software (Feature Extraction) provided by Agilent Technologies Inc. to give a signal value (processed signal).

Based on the above data, the signal ratios of the sense strand and the antisense strand were compared between the disease sample and a normal tissue, whereby the antisense RNA that is expressed disease-specifically was identified.

### Expression Analysis of Colorectal Cancer Sample

The expression of the sense strand and the antisense strand of CDK4 gene (cyclin-dependent kinase 4, GenBank ID: M14505), which is known to increase due to colorectal cancer, was analyzed according to the above-mentioned method. As the probe, a sequence complementary to SEQ ID NO: 1 was used for the antisense strand and the sequence of SEQ ID NO: 2 was used for the sense strand.

As the samples, cancer tissues obtained from surgery of six colorectal cancer patients, and the surrounding normal tissues thereof were used, and RNA was extracted by the guanidinium-thiocyanate-phenol-chloroform method using Trizol (Invitrogen) and the like.

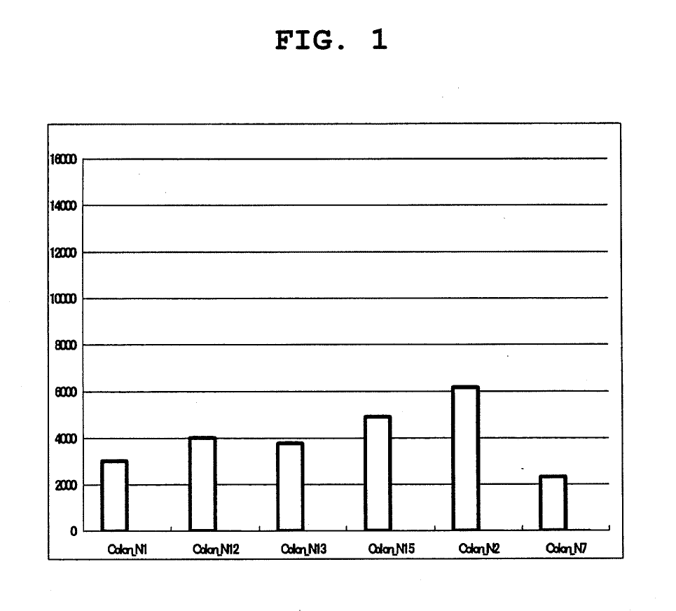

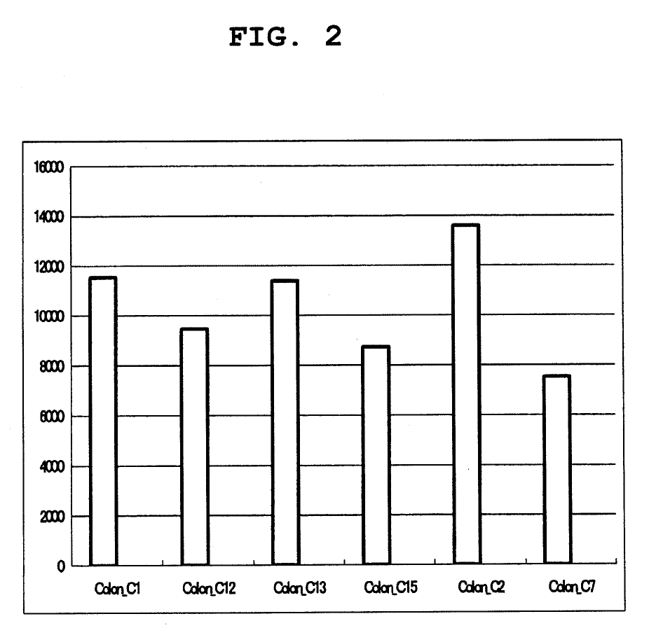

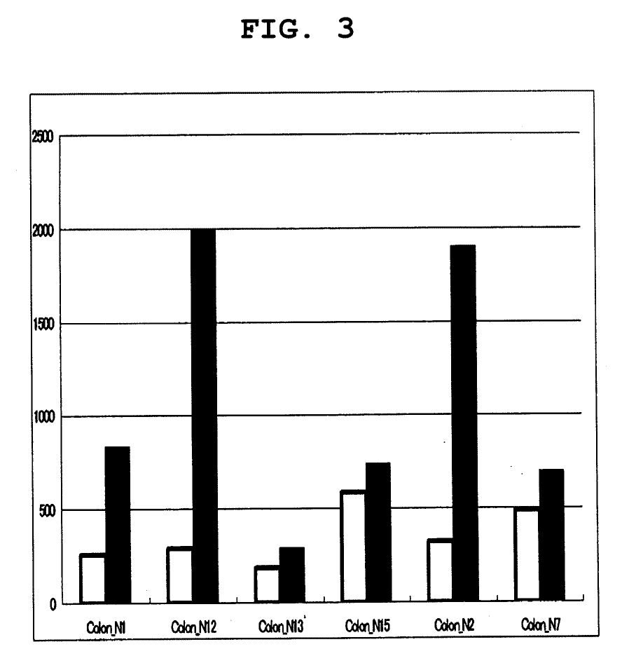

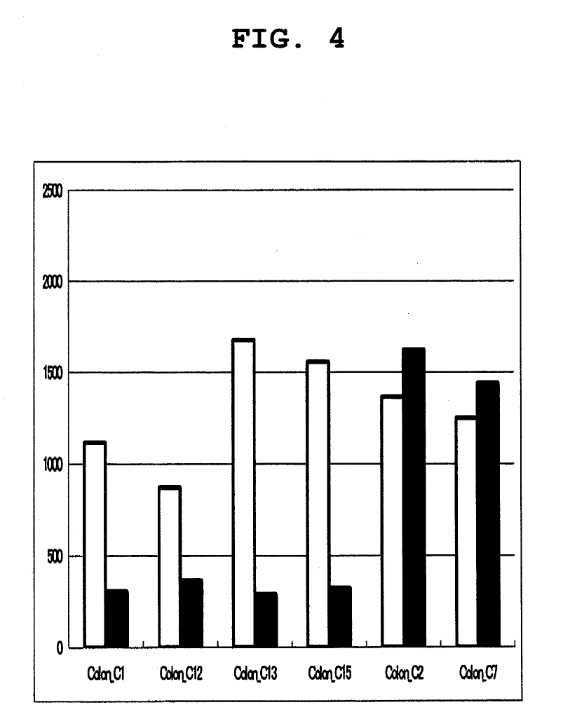

The obtained samples were labeled by the oligo(dT) priming method and analyzed. As a result, sense RNA showed generally higher expression in colorectal cancer samples than in normal tissues, but expression of antisense RNA was not detected (FIGS. 1 and 2). On the other hand, when the samples were labeled by the random priming method, antisense RNA showed higher expression than did the cancer gene (sense strand) in normal tissues (FIGS. 3 and 4). More interestingly, in 4 colorectal cancer samples out of 6 samples, the expression of antisense RNA was lower than that of cancer gene, and the expression of the sense strand and the antisense strand was reversed between normal and the cancer samples.

### Expression Analysis of Liver Cancer Sample

The expression of the sense strand and the antisense strand of MAPK7 gene (mitogen-activated protein kinase 7, GenBank ID: U25278), which is known to increase due to liver cancer, was analyzed according to the above-mentioned method as in the case of colorectal cancer. As the probe, a sequence complementary to SEQ ID NO: 3 was used for the antisense strand and the sequence of SEQ ID NO: 4 was used for the sense strand. As the analysis sample, the samples of five liver cancer patients were used.

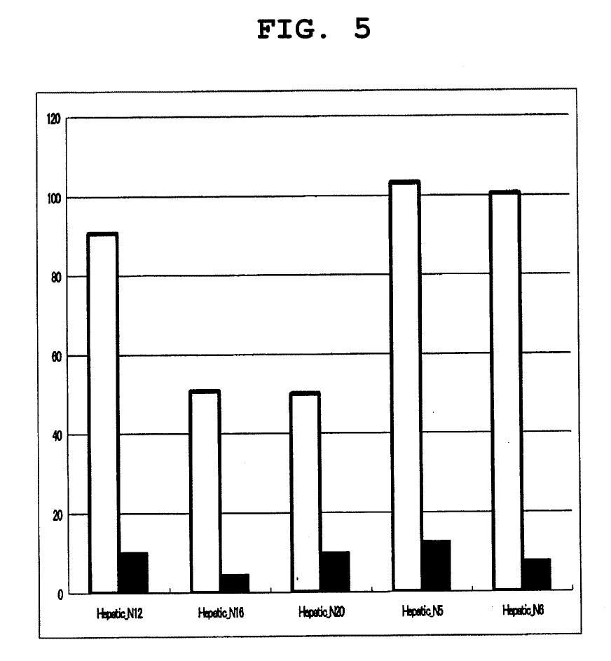

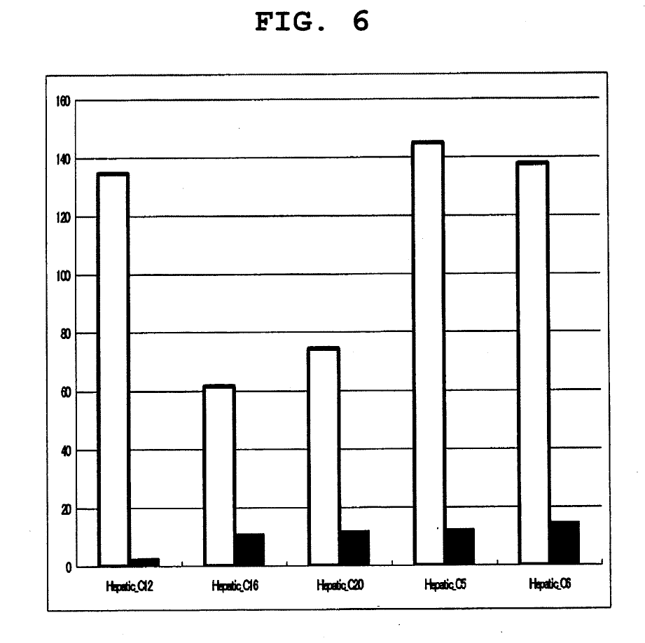

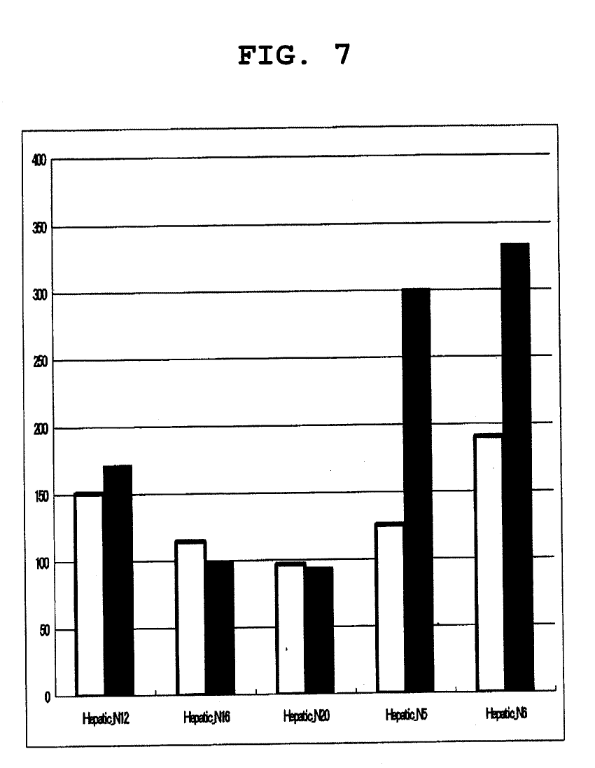

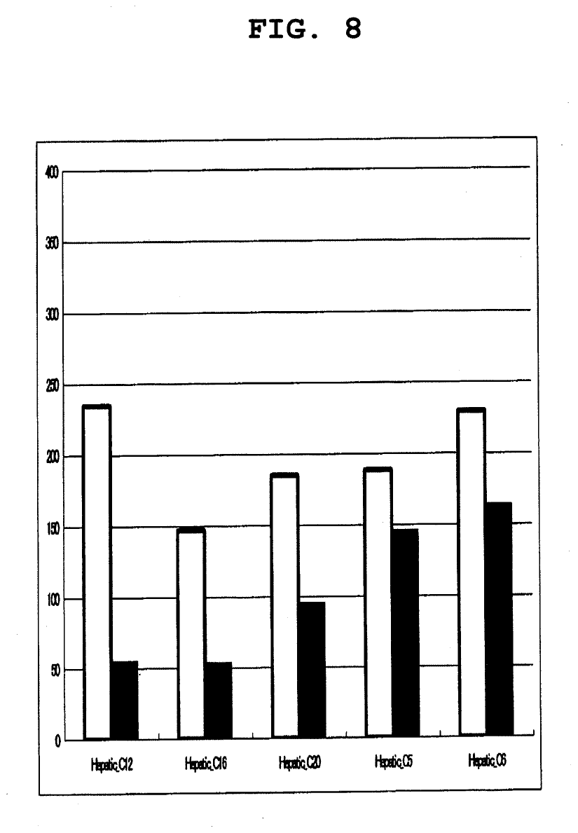

Similar to the colorectal cancer samples, the samples were labeled by the oligo(dT) priming method and analyzed. As a result, sense RNA showed generally higher expression in liver cancer samples than in normal tissues (FIGS. 5 and 6). On the other hand, when the samples were labeled by the random priming method, antisense RNA showed higher expression than did the cancer gene (sense strand) in normal tissues, and conversely, lower than did the cancer gene in the liver cancer samples. Thus, the expression of the sense strand and the antisense strand was reversed between normal tissues and the cancer samples (FIGS. 7 and 8).

The above results indicate that colorectal cancer and liver cancer patients show specific expression patterns in that the expression of sense strand and antisense strand of CDK4 and MAPK7 are reversed between normal tissue and cancer sample. It is strongly suggested therefrom that sense strand RNA and antisense strand RNA control expression of each other by an interaction through formation of double stranded RNA and the like.

### EXAMPLE 3

### Production of Array Containing 40000 or More Probe Sets (44 k Array)

In the same manner as in Example 1, human and mouse microarrays containing the following gene sets were produced.

### 1. Sense-Antisense Pair

As a result of cDNA sequence analysis, it is a pair of genes in a sense-antisense relationship.

human: 12306 genes (6153 pairs)

mouse: 15274 genes (7637 pairs)

### 2. Pair Based on Synteny Analysis

Gene of sense-antisense relationship in mouse, for which cDNA sequence of antisense strand has not been found in human (or vice versa). Probe is set for artificial detection of expression of antisense strand in region where antisense strand lacks cDNA sequence (Artificial Antisense Sequence: AFAS).

human: 10197 genes+10197 AFAS probes

mouse: 6774 genes+6774 AFAS probes

3. Non-Coding RNA (ncRNA) Gene Candidate

A gene not considered to encode a protein.

human: 5386 genes

mouse: 4459 genes

### 4. Cancer-Related Gene

Genes useful for cancer research. AFAS probe is set for strand opposite of gene, irrespective of whether endogenous antisense cDNA has been found.

human: 561 genes+2204 AFAS probes

mouse: 577 genes+2276 AFAS probes

### 5. Genome Imprinting Gene

Genome imprinting genes and candidates therefor, and AFAS probes thereof are contained.

human: 74 genes+447 AFAS probes

mouse: 88 genes+499 AFAS probes

6. Gene Considered to have No Antisense RNA

Gene predicted to show no expression of antisense strand, from cDNA information and CAGE data of RIKEN Genomic Sciences Center. At least two AFAS probes are contained for one gene.

human: 77 genes+152 AFAS probes

mouse: 1605 genes+3210 AFAS probes

### 7. Others, Containing Housekeeping Genes and Developmental Marker Genes.

About 30-40 genes for both human and mouse.

The number of each probe in the above-mentioned 1-6 and the total thereof, and the number of cDNAs utilized for designing AFAS probes are summarized in Table 1 for human and mouse.

TABLE 1
Number of designed probes (44K)

| | human | mouse |
|---|---|---|
| sense-antisense | 12,306 | 15,274 |
| pairs | | |
| pairs based on | 20,394 (10,197) | 13,548 (6,774) |
| synteny analysis | | |
| number of ncRNA | 5,386 | 4,459 |
| candidates | | |
| cancer related | 2,765 (561) | 2,853 (577) |
| genes | | |
| genome imprinting | 521 (74) | 587 (88) |
| genes | | |
| gene considered to | 229 (77) | 4,815 (1605) |
| have no antisense | | |
| transcription | | |
| product | | |
| total | 41,601 | 41,536 |

* Parentheses show the number of cDNAs used for setting AFAS sequence

### INDUSTRIAL APPLICABILITY

AFAS probe can be produced for not only the genes used for cancer research but also comprehensively for genes known to lack expression of antisense strand. Thus, when combined with labeling by a random priming method, biologically important novel antisense RNAs such as disease-specific antisense RNA and the like can be identified. Hence, it is useful for the development of a diagnostic method of diseases, search of drug discovery target and the like, based on the identified RNA.

1. A probe set comprising at least one kind of probe consisting of a nucleic acid comprising an artificial nucleotide sequence capable of hybridizing to an antisense strand sequence deduced from a known cDNA sequence.

2. The probe set of claim 1, wherein at least one kind of the cDNA is free of addition of a poly(A) chain when its antisense strand is transcribed.

3. The probe set of claim 1, wherein the cDNA is derived from a mammal.

4. The probe set of claim 3, wherein the mammal is a human or a mouse.

5. The probe set of claim 1, further comprising a probe consisting of a nucleic acid comprising a nucleotide sequence capable of hybridizing to a sense strand of the cDNA.

6. The probe set of claim 1, wherein the number of the cDNA is 100 or more.

7. A microarray comprising a substrate and the probe set of claim 1 immobilized thereon.

8. A method of detecting an endogenous antisense RNA in an RNA-containing sample using the microarray of claim 7, which comprises the steps of
(a) labeling RNA in the sample by random priming,
(b) contacting each probe on the microarray with said labeled RNA,
(c) washing away the RNA not bound to the probe, and
(d) detecting the label of the RNA bound to the probe.

9. The method of claim 8, comprising detecting the expression of mRNA transcribed from a sense strand corresponding to the antisense RNA.

10. The method of claim 8, wherein the sample is derived from a mammal.

11. The method of claim 10, wherein the mammal is a human or a mouse.

12. The method of claim 9 used for comparison of expression patterns of endogenous antisense RNAs of mammals, comprising subjecting a target animal and a control animal to said steps (a)-(d), and further identifying endogenous antisense RNA showing different expression patterns between the target animal and the control animal, by comparing the obtained expression patterns of endogenous antisense RNAs of the target animal and that obtained of the control animal.

13. The method of claim 12, wherein the target animal is affected with a given disease, or an animal model of a given disease.
